(19) United States
(12) Patent Application Publication
(10) Pub. No.: US 2020/0281770 A1
(43) Pub. Date: Sep. 10, 2020

(54) COMBINATION SLEEP EYE MASK AND FACE MASK

(71) Applicants: Manuel Salvador Moreno, Hollywood, FL (US); Edgar Daniel Bosquez Fuentes, Hollywood, FL (US)

(72) Inventors: Manuel Salvador Moreno, Hollywood, FL (US); Edgar Daniel Bosquez Fuentes, Hollywood, FL (US)

(21) Appl. No.: 16/291,857

(22) Filed: Mar. 4, 2019

(51) Int. Cl. A61F 9/04

(52) U.S. Cl. CPC A61F 9/04

(57) ABSTRACT

A combination sleep eye mask and face mask for covering both the eyes and mouth for improved sleeping conditions and privacy in public includes an eye mask body having an outer side, an inner side, an eye portion, a right extension, and a left extension. A first attachment mechanism is coupled to each of the right extension and the left extension and is selectively engageable to secure the eye mask body around a user's head. A face mask body has an outside surface, an inside surface, a right end, and a left end. The face mask body is configured to cover from below the user's nose to below the user's chin. A second attachment mechanism is coupled to each of the right end and the left end and is selectively engageable with the eye mask body to secure the face mask body below the eye mask body.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to sleep masks and more particularly pertains to a new sleep mask for covering both the eyes and mouth for improved sleeping conditions and privacy in public.

### BRIEF SUMMARY OF THE INVENTION

An embodiment of the disclosure meets the needs presented above by generally comprising an eye mask body having an outer side, an inner side, an eye portion, a right extension, and a left extension. A first attachment mechanism is coupled to each of the right extension and the left extension and is selectively engageable to secure the eye mask body around a user's head. A face mask body has an outside surface, an inside surface, a right end, and a left end. The face mask body is configured to cover from below the user's nose to below the user's chin. A second attachment mechanism is coupled to each of the right end and the left end and is selectively engageable with the eye mask body to secure the face mask body below the eye mask body.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front elevation view of a combination sleep eye mask and face mask according to an embodiment of the disclosure.

FIG. 2 is a rear elevation view of an embodiment of the disclosure.

FIG. 3 is an in-use view of an embodiment of the disclosure.

FIG. 4 is an isometric view of an embodiment of the disclosure.

### DETAILED DESCRIPTION OF THE INVENTION

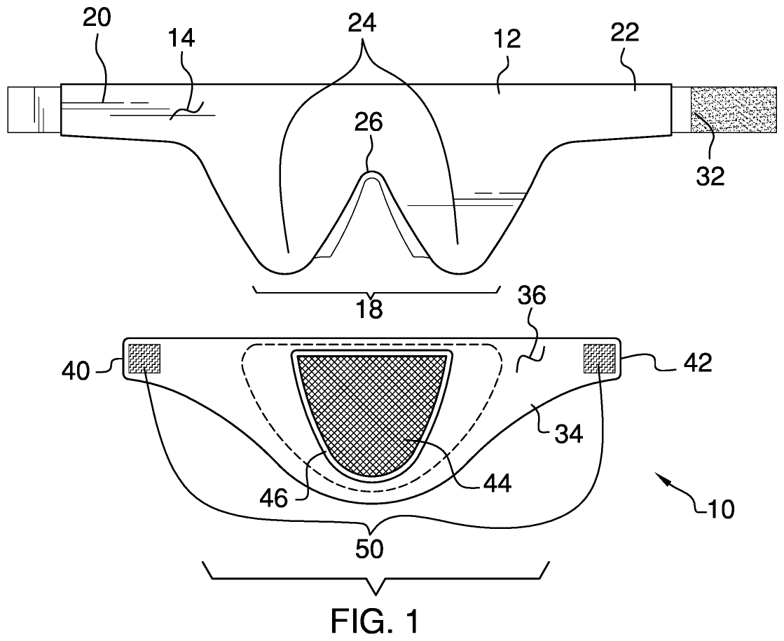

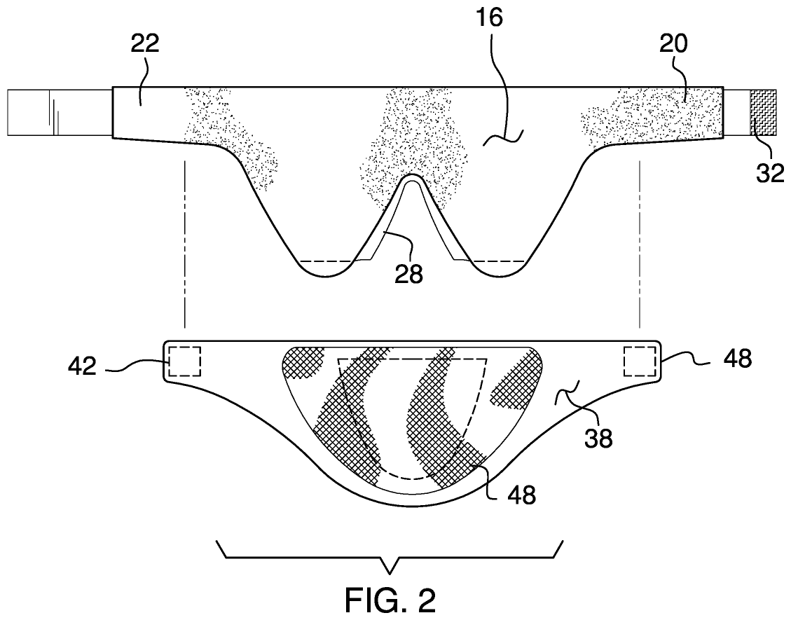

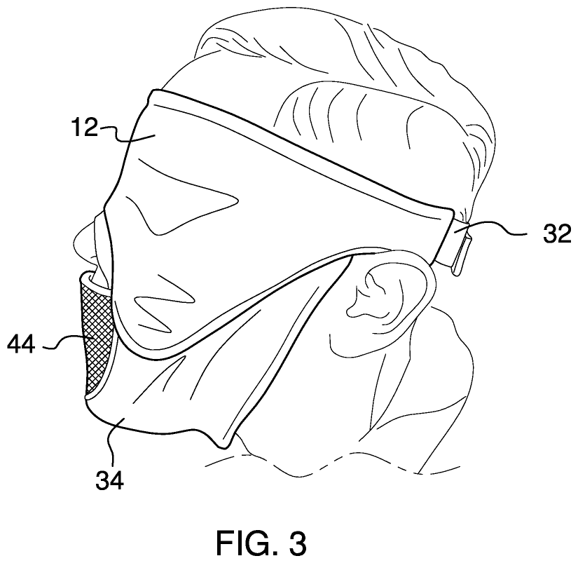

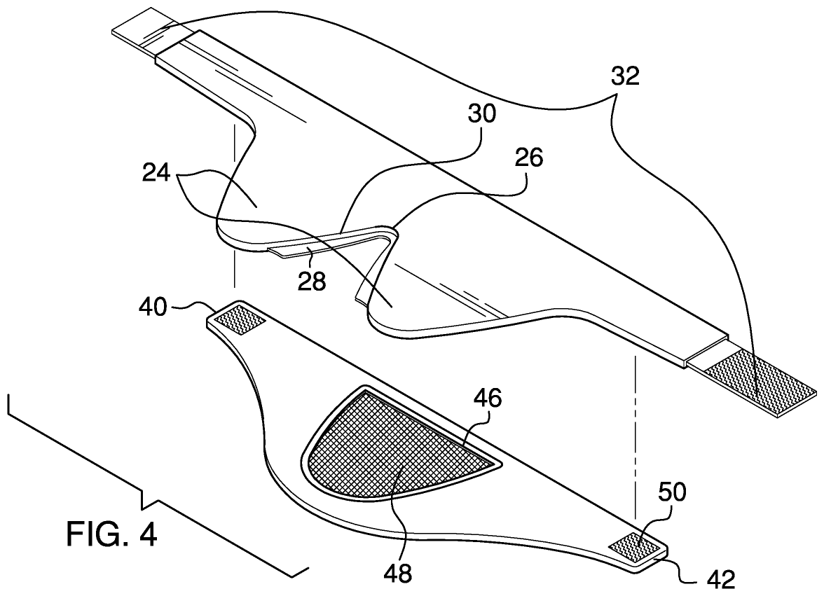

With reference now to the drawings, and in particular to FIGS. 1 through 4 thereof, a new sleep mask embodying the principles and concepts of an embodiment of the disclosure and generally designated by the reference numeral 10 will be described.

As best illustrated in FIGS. 1 through 4, the combination sleep eye mask and face mask 10 generally comprises an eye mask body 12 having an outer side 14, an inner side 16, an eye portion 18, a right extension 20, and a left extension 22. The eye portion 18 may comprise a pair of eye extensions 24 each configured to extend beneath a user's eyes. The pair of eye extensions 24 forms a nose cutout 26 therebetween. A nose flap 28 may be coupled to an inner edge 30 of the nose cutout to aid in forming a light seal around the user's nose. A first attachment mechanism 32 is coupled to each of the right extension 20 and the left extension 22. The first attachment mechanism 32 is selectively engageable and is configured to secure the eye mask body 12 around the user's head. The first attachment mechanism 32 may be a hook- and loop fastener. Alternatively, the right extension 20 and the left extension 22 may be permanently adhered or sewn and elasticized to create an adjustable headband.

A face mask body 34 has an outside surface 36, an inside surface 38, a right end 40, and a left end 42. The face mask body 34 has a mouth aperture 44 extending from the outside surface 36 through the inside surface 38. The mouth aperture 44 may be parabolic and is configured to rest in front of the user's mouth for improved breathability. The outside surface 36 may have a reinforced edging 46 around the perimeter of the mouth aperture 44. The face mask body 34 is configured to cover from below the user's nose to below the user's chin. A mesh screen 48 is coupled to the face mask body 34. The mesh screen 48 may also be parabolic and is coupled to the inside surface 38 covering the mouth aperture 44 and extending beyond the perimeter of the mouth aperture in all directions to hide the user's mouth while maintaining breathability. A second attachment mechanism 50 is coupled to each of the right end 40 and the left end 42. The second attachment mechanism 50 is selectively engageable with the inner side 16 of the eye mask body to secure the face mask body 34 below the eye mask body 12. The second attachment mechanism 50 may be a hook-and-loop fastener. Each of the eye mask body 12 and the face mask body 34 may be elasticized to create a snug fit on the user's face.

In use, the first attachment mechanism 32 is used to secure the eye mask body 12 around the user's head to cover her eyes. The second attachment mechanism 50 is the used to attach the face mask body 34 and cover the user's mouth. The user may then comfortably and privately sleep in public places, such as on planes, without worrying about the embarrassment of a gaping mouth.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A combination sleep eye mask and face mask comprising:
an eye mask body, the eye mask body having an outer side, an inner side, an eye portion, a right extension, and a left extension;
a first attachment mechanism coupled to the eye mask body, the attachment mechanism being coupled to each of the right extension and the left extension, the first attachment mechanism being selectively engageable and configured to secure the eye mask body around a user's head;
a face mask body, the face mask body having an outside surface, an inside surface, a right end, and a left end, the face mask body being configured to cover from below the user's nose to below the user's chin; and
a second attachment mechanism coupled to the face mask body, the second attachment mechanism being coupled to each of the right end and the left end, the second attachment mechanism being selectively engageable with the eye mask body to secure the face mask body below the eye mask body.

2. The combination sleep eye mask and face mask of claim 1 further comprising the face mask body having a mouth aperture extending from the outside surface through the inside surface, the mouth aperture being configured to rest in front of the user's mouth for improved breathability.

3. The combination sleep eye mask and face mask of claim 2 further comprising a mesh screen coupled to the face mask body, the mesh screen covering the mouth aperture to hide the user's mouth while maintaining breathability.

4. The combination sleep eye mask and face mask of claim 3 further comprising the mesh screen being coupled to the inside surface and extending beyond the perimeter of the mouth aperture in all directions.

5. The combination sleep eye mask and face mask of claim 2 further comprising the outside surface of the face mask body having a reinforced edging around the perimeter of the mouth aperture.

6. The combination sleep eye mask and face mask of claim 3 further comprising each of the mouth aperture and the mesh screen being parabolic.

7. The combination sleep eye mask and face mask of claim 1 further comprising the eye portion comprising a pair of eye extensions, each of the pair of eye extensions being configured to extend beneath the user's eyes, the pair of eye extensions forming a nose cutout therebetween.

8. The combination sleep eye mask and face mask of claim 7 further comprising each of the pair of eye extensions being parabolic.

9. The combination sleep eye mask and face mask of claim 7 further comprising a nose flap coupled to the eye mask body, the nose flap being coupled to an inner edge of the nose cutout to aid in forming a light seal around the user's nose.

10. A combination sleep eye mask and face mask comprising:
an eye mask body, the eye mask body having an outer side, an inner side, an eye portion, a right extension, and a left extension, the eye portion comprising a pair of eye extensions each being configured to extend beneath a user's eyes, the pair of eye extensions forming a nose cutout therebetween;
a nose flap coupled to the eye mask body, the nose flap being coupled to an inner edge of the nose cutout to aid in forming a light seal around the user's nose;
a first attachment mechanism coupled to the eye mask body, the attachment mechanism being coupled to each of the right extension and the left extension, the first attachment mechanism being selectively engageable and configured to secure the eye mask body around the user's head;
a face mask body, the face mask body having an outside surface, an inside surface, a right end, and a left end, the face mask body having a mouth aperture extending from the outside surface through the inside surface, the mouth aperture being parabolic and configured to rest in front of the user's mouth for improved breathability, the outside surface having a reinforced edging around the perimeter of the mouth aperture, the face mask body being configured to cover from below the user's nose to below the user's chin;
a mesh screen coupled to the face mask body, the mesh screen being parabolic and coupled to the inside surface covering the mouth aperture and extending beyond the perimeter of the mouth aperture in all directions to hide the user's mouth while maintaining breathability; and
a second attachment mechanism coupled to the face mask body, the second attachment mechanism being coupled to each of the right end and the left end, the second attachment mechanism being selectively engageable with the eye mask body to secure the face mask body below the eye mask body.

11. The combination sleep eye mask and face mask of claim 1 further comprising each of the first attachment mechanism and the second attachment mechanism being hook-and-loop fasteners, the second attachment mechanism being selectively engageable with the inner side of the eye mask body.

12. The combination sleep eye mask and face mask of claim 10 further comprising each of the first attachment mechanism and the second attachment mechanism being hook-and-loop fasteners, the second attachment mechanism being selectively engageable with the inner side of the eye mask body.

13. The combination sleep eye mask and face mask of claim 1 further comprising each of the eye mask body and the face mask body being elasticized to create a snug fit on the user's face.

14. The combination sleep eye mask and face mask of claim 10 further comprising each of the eye mask body and the face mask body being elasticized to create a snug fit on the user's face.

15. A combination sleep eye mask and face mask comprising:
an eye mask body, the eye mask body having an outer side, an inner side, an eye portion, a right extension, and a left extension, the eye portion comprising a pair of eye extensions each being configured to extend beneath a user's eyes, the pair of eye extensions forming a nose cutout therebetween;
a nose flap coupled to the eye mask body, the nose flap being coupled to an inner edge of the nose cutout to aid in forming a light seal around the user's nose;
a first attachment mechanism coupled to the eye mask body, the attachment mechanism being coupled to each of the right extension and the left extension, the first attachment mechanism being selectively engageable hook-and-loop fasteners configured to secure the eye mask body around the user's head;
a face mask body, the face mask body having an outside surface, an inside surface, a right end, and a left end, the face mask body having a mouth aperture extending from the outside surface through the inside surface, the mouth aperture being parabolic and configured to rest in front of the user's mouth for improved breathability, the outside surface having a reinforced edging around the perimeter of the mouth aperture, the face mask body being configured to cover from below the user's nose to below the user's chin;
a mesh screen coupled to the face mask body, the mesh screen being parabolic and coupled to the inside surface covering the mouth aperture and extending beyond the perimeter of the mouth aperture in all directions to hide the user's mouth while maintaining breathability; and
a second attachment mechanism coupled to the face mask body, the second attachment mechanism being coupled to each of the right end and the left end, the second attachment mechanism being selectively engageable with the inner side of the eye mask body to secure the face mask body below the eye mask body, the second attachment mechanism being a hook-and-loop fastener;
wherein each of the eye mask body and the face mask body is elasticized to create a snug fit on the user's face.
